SEQUENCE LISTING (4 sequences)

SEQ ID NO: 1 - DNA, 1254 bp, Mus sp.
  CDS (53)..(979)
agcctataaagcacgggcactggcgggagacgtgcactgaccgaccgtggtaatggaccagcacacacttgatgtggaggataccgcggatgccagacatccagcaggtacttcgtgcccctcggatgcggcgctcctcagagataccgggctcctcgcggacgctgcgctcctctcagatactgtgcgccccacaaatgccgcgctccccacggatgctgcctaccctgcggttaatgttcgggatcgcgaggccgcgtggccgcctgcactgaacttctgttcccgccacccaaagctctatggcctagtcgctttggttttgctgcttctgatcgccgcctgtgttcctatcttcacccgcaccgagcctcggccagcgctcacaatcaccacctcgcccaacctgggtacccgagagaataatgcagaccaggtcacccctgtttcccacattggctgccccaacactacacaacagggctctcctgtgttcgccaagctactggctaaaaaccaagcatcgttgtgcaatacaactctgaactggcacagccaagatggagctgggagctcatacctatctcaaggtctgaggtacgaagaagacaaaaaggagttggtggtagacagtcccgggctctactacgtatttttggaactgaagctcagtccaacattcacaaacacaggccacaaggtgcagggctgggtctctcttgttttgcaagcaaagcctcaggtagatgactttgacaacttggccctgacagtggaactgttcccttgctccatggagaacaagttagtggaccgttcctggagtcaactgttgctcctgaaggctggccaccgcctcagtgtgggtctgagggcttatctgcatggagcccaggatgcatacagagactgggagctgtcttatcccaacaccaccagctttggactctttcttgtgaaacccgacaacccatgggaatgagaactatccttcttgtgactcctagttgctaagtcctcaagctgctatgttttatggggtctgagcaggggtcccttccatgactttctcttgtctttaactggacttggtatttattctgagcatagctcagacaagactttatataattcactagatagcattagtaaactgctgggcagctgctagataaaaaaaaatttctaaatcaaagtttatatttatattaatatataaaaataaatgtgtttgtaaataaaaaaaaaaaaaaa

SEQ ID NO: 2 - protein, 309 aa, Mus sp.
MDQHTLDVEDTADARHPAGTSCPSDAALLRDTGLLADAALLSDTVRPTNAALPTDAAYPAVNVRDREAAWPPALNFCSRHPKLYGLVALVLLLLIAACVPIFTRTEPRPALTITTSPNLGTRENNADQVTPVSHIGCPNTTQQGSPVFAKLLAKNQASLCNTTLNWHSQDGAGSSYLSQGLRYEEDKKELVVDSPGLYYVFLELKLSPTFTNTGHKVQGWVSLVLQAKPQVDDFDNLALTVELFPCSMENKLVDRSWSQLLLLKAGHRLSVGLRAYLHGAQDAYRDWELSYPNTTSFGLFLVKPDNPWE

SEQ ID NO: 3 - DNA, 1618 bp, Homo sapiens
  CDS (4)..(765)
gtcatggaatacgcctctgacgcttcactggaccccgaagccccgtggcctcccgcgccccgcgctcgcgcctgccgcgtactgccttgggccctggtcgcggggctgctgctgctgctgctgctcgctgccgcctgcgccgtcttcctcgcctgcccctgggccgtgtccggggctcgcgcctcgcccggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaaaacgcccagcctgggtgcagcccacctggacagagtccgaatcctactccatccttcatggagacccctggtgctgggtccctgctgctttctctacctcaaggggcttggcaggggtccctgctgctgacctccccttgaggaccctcctcacccactccttccccaagttggaccttgatatttattctgagcctgagctcagataatatattatatatattatatatatatatatatttctatttaaagaggatcctgagtttgtgaatggacttttttagaggagttgttttggggggggggtcttcgacattgccgaggctggtcttgaactcctggacttagacgatcctcctgcctcagcctcccaagcaactgggattcatcctttctattaattcattgtacttatttgcctatttgtgtgtattgagcatctgtaatgtgccagcattgtgcccaggctagggggctatagaaacatctagaaatagactgaaagaaaatctgagttatggtaatacgtgaggaatttaaagactcatccccagcctccacctcctgtgtgatacttgggggctagcttttttctttctttcttttttttgagatggtcttgttctgtcaaccaggctagaatgcagcggtgcaatcatgagtcaatgcagcctccagcctcgacctcccgaggctcaggtgatcctcccatctcagcctctcgagtagctgggaccacagttgtgtgccaccacacttggctaactttttaatttttttgcggagacggtattgctatgttgccaaggttgtttacatgccagtacaatttataataaacactcatttttcc

SEQ ID NO: 4 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

(19) United States
(12) Patent Application Publication
(10) Pub. No.: US 2003/0223989 A1
(43) Pub. Date: Dec. 4, 2003

(54) CD137 agonists to treat patients with IgE-mediated conditions

(76) Inventor: John D. Pluenneke, Parkville, MO (US)

Correspondence Address: IMMUNEX CORPORATION, LAW DEPARTMENT, 51 UNIVERSITY STREET, SEATTLE, WA, 98101

(21) Appl. No.: 10/420,050

(22) Filed: Apr. 18, 2003

Related U.S. Application Data
(60) Provisional application No. 60/373,536, filed on Apr. 18, 2002.

(51) Int. Cl. A61K 39/395; C12N 5/06

(52) U.S. Cl. 424/131.1; 424/144.1; 435/327

(57) ABSTRACT

There are disclosed methods for treating conditions mediated by IgE, comprising administering a CD137 agonist to a mammal afflicted with such a condition. CD137 agonists include CD137 ligand (CD137L) and agonistic antibodies to CD137; mammals to be treated include humans. Conditions mediated by IgE include asthma, atopic dermatitis, and allergy. CD137 agonists are also useful for treating conditions characterized by delayed eosinophil apoptosis, including nasal polyps and hypereosinophilic syndrome. Patients to be treated may be afflicted with, or at risk for, one or more of these conditions.

### CROSS-REFERENCE TO RELATED APPLICATIONS

This application claims the benefit of U.S. provisional patent application Serial No. 60/373,536, filed Apr. 18, 2002. The entire disclosure of the application is relied upon and incorporated by reference herein.

### BACKGROUND OF THE INVENTION

The Tumor Necrosis Factor/Nerve Growth Factor Receptor family of receptors includes two different TNF receptors (Type I and Type II; Smith et al., Science 248:1019, 1990 and Schall et al., Cell 61:361, 1990); nerve growth factor receptor (Johnson et al., Cell 47:545, 1986); B cell antigen CD40 (Stamenkovic et al., EMBO J. 8:1403, 1989); T cell antigen OX40 (Mallett et al., EMBO J. 9:1063, 1990); human Fas antigen (Itoh et al., Cell 66:233, 1991); and the inducible T cell surface receptor 4-1BB (Kwon et al., Cell. Immunol. 121:414, 1989; Schwartz et al., Gene 134:295, 1993; Alderson et al., Eur. J. Immunol 24:2219, 1994), the human form of which is now referred to as CD137.

CD137 binds with high affinity to a ligand present on antigen-presenting cells, and subsequently interacts with TNF receptor-associated factors (TRAFs) to induce NFkappaB (Arch and Thompson, Mol. Cell. Biol. 18:558, 1998). The ligand for CD137 (CD137L; also referred to as 4-1BBL) is described in U.S. Pat. No. 5,674,704, issued Oct. 17, 1997. As shown therein, CD137L is a type II extracellular membrane polypeptide with an intracellular (cytoplasmic) domain at the N-terminus of the polypeptide, followed by a transmembrane region, and an extracellular (receptor-binding) domain at the C-terminus of the polypeptide. Soluble CD137L polypeptides may be derived from the extracellular domain, as described in U.S. Pat. No. 5,674,704, and herein. CD137 is inducible in splenocytes and T cells; the ligand, whose expression is also inducible, is found primarily on antigen presenting cells (Vinay and Kwon, Sem. Immunol. 10:481 1998). Expression of CD137 has also been detected in monocytes, some transformed cells and eosinophils after activation (Schwarz et al., Blood 85:1043, 1995; Heinisch et al., J. Allergy Clin. Immunol. 108:21, 2001).

The CD137/CD137L interaction appears to play diverse, and sometimes apparently contradictory, roles in various aspects of an immune response. It appears to be important in inhibiting activation-induced cell death in T cells (Hurtado et al., J. Immunol. 158:2600, 1997), but abrogates anti-apoptotic effects of other cytokines in neutrophils (Heinisch et al., Eur. J. Immunol. 30:3441, 2001). CD137 thus may play a role in immune function homeostasis (Ebata et al., Eur. J. Immunol. 31:1210, 2001) and may represent a target costimulatory system that can be targeted in treatment of cancer or the inflammatory response (Blazar et al., J. Immunol. 166:174, 2001; Takahashi et al., Immunol. Lett. 76:183, 2001; Kim and Broxmeyer, J. Hematother. Stem Cell Res. 10:441, 2001; Kim et al., Cancer Res. 61:2031, 2001).

IgE-mediated allergic disorders include asthma, atopic dermatitis and allergy. The etiology of these disorders is complex, but is generally believed to include stimulation of an immune response against an allergen, which is modulated primarily along the T_{H}2 pathway. Numerous cytokines and other agents have been implicated in the development of allergic disorders, including Interleukins 4, 5, 9 and 13 (Renauld, J. C., J. Clin. Pathol. 54:577, 2001; Lee et al., J. Allergy Clin. Immunol. 107:945, 2001). There is extensive interest in developing therapeutic agents that modulate cytokines and other mediators of atopic disease for treatment of these conditions (Alvarez et al., Curr. Pharm. Des. 7:1059, 2001; Barnes, P. J., J. Allergy Clin. Immunol. 108:S72, 2001).

### SUMMARY OF THE INVENTION

The present invention provides a method for treating a condition mediated by IgE, comprising administering a CD137 agonist to a mammal afflicted with such a condition. In one embodiment, the CD137 agonist is selected from the group consisting of CD137 ligand (CD137L) and agonistic antibodies to CD137. In a particular embodiment the mammal is a human. Conditions mediated by IgE include asthma, atopic dermatitis, allergy, and combinations thereof.

Additional conditions for which treatment with a CD137 agonist will be useful include conditions characterized by the presence of eosinophils expressing functional CD137. Such conditions include delayed eosinophil apoptosis and/or accumulation or elevated numbers of eosinophils, including nasal polyps, hyper-IgE syndrome, and hypereosinophilic syndrome.

Examples of useful CD137L polypeptides are selected from the group consisting of: (a) a polypeptide comprising amino acids x through y of SEQ ID NO:2, wherein x is selected from the group consisting of amino acids 104, 105, 106, 107, 108 and 109 of SEQ ID NO:2, and y is selected from the group consisting of amino acids 304, 305, 306, 307, 308 and 309 of SEQ ID NO:2; (b) a polypeptide comprising amino acids x through y SEQ ID NO:4, where x is selected from the group consisting of amino acids 49, 50, 51, 52, 53, and 54 of SEQ ID NO:4 and y is selected from the group consisting of amino acids 249, 250, 251, 252, 253 and 254 of SEQ ID NO:4; (c) CD137L polypeptides that are at least about 80% identical in amino acid sequence to the polypeptides of (a) or (b); and (d) fragments of the aforementioned CD137L polypeptides that are CD137 agonists.

Examples of useful agonistic antibodies to CD137 are selected from the group consisting of: an antibody produced by hybridoma cell line m4-1BBm6, deposited with the American Type Culture Collection, in Manassas, Va. on Nov. 28, 2001 and given accession number PTA-3885; and derivatives and mutants of the aforementioned antibodies, including scFv, Fab, F(ab′)2, diabodies, triabodies, IgA, IgG1, IgG2, IgG3, IgG4, IgM, IgE, IgD, and IgG4 having a mutation in a hinge region that alleviates a tendency to form intra-H chain disulfide bonds.

Also provided are methods wherein the CD137 agonist is co-administered with an agent selected from the group consisting of an IgE antagonist, a cytokine antagonist, a corticosteroid, a beta agonist, a leukotriene antagonist, a xanthine, fluticasone, salmeterol, albuterol, a non-steroidal agent such as cromolyn, and combinations thereof. Useful agents include cytokine antagonists that antagonize a cytokine selected from the group consisting of IL-4, IL-5, IL-9, IL-13, and/or combinations thereof, as well as an anti-IgE antibody such as a humanized antibody (for example, Xolair™). Thus, a CD137 agonist and another agent or agents may be administered concurrently, consecutively, or in the form of a single composition comprising two or more agents as active ingredients.

The CD137 agonist(s) (and any agents administered therewith) may be administered in the form of a composition that additionally comprises a diluent, excipient, or carrier, which composition(s) provide another embodiment of the invention. Also provided are kits for use by a medical practitioner, comprising a CD137 agonist as described above and a label or other instructions for use in treating a condition mediated by IgE. Such kits may optionally include an agent (such as those described previously) for co-administration with a CD137 agonist.

### DETAILED DESCRIPTION OF THE INVENTION

The present invention provides methods for treating or preventing conditions mediated by IgE, and for inducing biological activities of CD137 (4-1BB/ILA) in vivo. One method comprises administering a CD137 agonist to a patient afflicted with such a condition. A CD137 agonist is a molecule that is capable of binding CD137 and causing a signal to be transduced thereby. Exemplary CD137 agonists include CD137 ligand (CD137L) and agonistic antibodies to CD137. Compositions for use in such methods for treating certain conditions also are provided.

Among the conditions to be treated in accordance with the present invention are IgE-mediated allergic disorders, including asthma, atopic dermatitis, eczema and pollinosis as well as allergy (including allergic rhinitis) and anaphylactic shock.

CD137 may also play a role in the delayed eosinophil apoptosis observed in nasal polyps and hypereosinophilic syndrome; accordingly, these conditions may also be treated using the present invention. Accordingly, CD137 agonists will be useful in treating or preventing conditions characterized by the presence of eosinophils expressing functional CD137. Additional conditions for which the instant inventive methods may prove of use include septic/reactive arthritis, dermatitis herpetiformis, chronic idiopathic urticaria, scleroderma, hypertrophic scarring, Whipple's Disease, benign prostate hyperplasia, Churg-Strauss syndrome, hyper IgE syndrome, and lung and/or other disorders in which IgE and/or eosinophils play a role, including conditions in which inflammatory cytokines such as IL-1 and/or TNF play a role.

CD137 Ligand

CD137L and the murine ortholog thereof (4-1BBL) are disclosed in U.S. Pat. No. 5,674,704, the disclosure of which is incorporated by reference herein. The nucleotide sequence of murine 4-1BBL cDNA and the amino acid sequence encoded thereby are presented in SEQ ID NO:1 and SEQ ID NO:2, respectively. Murine 4-1BBL protein comprises a cytoplasmic domain (amino acids 1-82 of SEQ ID NO:2), a transmembrane region (amino acids 83-103), and an extracellular domain (amino acids 104-309). The nucleotide sequence of a human CD137L (4-1BBL) cDNA and the amino acid sequence encoded thereby are presented in SEQ ID NO:3 and SEQ ID NO:4, respectively. This human 4-1BB-L protein comprises a cytoplasmic domain (amino acids 1-25 of SEQ ID NO:4), a transmembrane region (amino acids 26-48), and an extracellular domain (amino acids 49-254).

Soluble forms of CD137L will be useful in the present invention. Examples of soluble polypeptides include those comprising the entire extracellular domain. Representative examples of such soluble forms include, but are not limited to (a) a polypeptide comprising amino acids x through y of SEQ ID NO:2, wherein x is selected from the group consisting of amino acids 104, 105, 106, 107, 108 and 109 of SEQ ID NO:2, and y is selected from the group consisting of amino acids 304, 305, 306, 307, 308 and 309 of SEQ ID NO:2; and (b) a polypeptide comprising amino acids x through y SEQ ID NO:4, where x is selected from the group consisting of amino acids 49, 50, 51, 52, 53, and 54 of SEQ ID NO:4 and y is selected from the group consisting of amino acids 249, 250, 251, 252, 253 and 254 of SEQ ID NO:4.

Moreover, fragments of the aforesaid soluble forms of CD137L that retain the ability to bind CD137 and transduce a signal thereby will also be useful in the inventive methods. Like other members of the TNFR/NGFR family, the extracellular region of CD137 is made up of a ligand-binding domain and a ‘spacer’ region of about 30 to 32 amino acids between the transmembrane region and the ligand binding domain. Examples of fragments of soluble CD137 include polypeptides from which the spacer region has been removed. Representative examples of such fragments include, but are not limited to: (a) a polypeptide comprising amino acids x through y of SEQ ID NO:2, wherein x is selected from the group consisting of amino acids 134, 135, 136, 137, 138, 139, 140, 141, 142, and 143 of SEQ ID NO:2, and y is selected from the group consisting of amino acids 304, 305, 306, 307, 308 and 309 of SEQ ID NO:2; and (b) a polypeptide comprising amino acids x through y SEQ ID NO:4, where x is selected from the group consisting of amino acids 79, 80, 81, 82, 83, 84, 85, 86, 87, and 88 of SEQ ID NO:4 and y is selected from the group consisting of amino acids 249, 250, 251, 252, 253 and 254 of SEQ ID NO:4.

Additional soluble forms of CD137 include those in which all or a portion of the transmembrane region has removed, for example a polypeptide comprising all or a portion of the cytoplasmic domain of CD137 and all or a portion of the extracellular domain of CD137. By ‘removed’ is meant that one or more of the amino acids that form the transmembrane region is/are deleted, or substituted with amino acids that do not have the property of remaining within a cell membrane (i.e., they are not hydrophobic). A polypeptide lacking a transmembrane region by such deletion or substitution of amino acid(s) will not remain associated with the cell membrane.

Such fragments can be prepared by methods that are well-known in the art for preparing deletion mutants and tested for the ability to bind CD137 and transduce a signal thereby, substantially as described herein (or by using other methods of assessing CD137L biological activity that are known in the art). For example, a DNA fragment encoding a desired polypeptide fragment may be subcloned into an expression vector. Alternatively, a desired DNA sequence may be chemically synthesized using known techniques. DNA fragments also may be produced by restriction endonuclease digestion of a full length cloned DNA sequence, and isolated by electrophoresis on agarose gels. Linkers containing restriction endonuclease cleavage site(s) may be employed to insert the desired DNA fragment into an expression vector, or the fragment may be digested at cleavage sites naturally present therein. The well known polymerase chain reaction procedure also may be employed to isolate a DNA sequence encoding a desired protein fragment by using oligonucleotide primers comprising sequences that define the termini of the desired fragment.

In another approach, enzymatic treatment (e.g., using Bal 31 exonuclease) may be employed to delete terminal nucleotides from a DNA fragment to obtain a fragment having a particular desired terminus. Among the commercially available linkers are those that can be ligated to the blunt ends produced by Bal 31 digestion, and which contain restriction endonuclease cleavage site(s). Alternatively, oligonucleotides that reconstruct the N- or C-terminus of a DNA fragment to a desired point may be synthesized. The oligonucleotide may contain a restriction endonuclease cleavage site upstream of the desired coding sequence and position an initiation codon (ATG) at the N-terminus of the coding sequence. The well known polymerase chain reaction procedure also may be employed to isolate a DNA sequence encoding a desired protein fragment by using oligonucleotide primers comprising sequences that define the termini of the desired fragment.

Oligomeric (multimeric) forms of CD137L are encompassed by the present invention. The oligomers may be dimers or trimer, or higher-order multimers. Separate polypeptide chains may be joined by interchain disulfide bonds formed between cysteine residues to form oligomers. Alternatively, the multimers may be expressed as fusion proteins, with or without spacer amino acids between the protein moieties, using recombinant DNA techniques. In one embodiment of the present invention, two or three soluble CD137L polypeptides are joined via a polypeptide linker (e.g., an antibody-derived or peptide linker as described below).

In one embodiment of the present invention, a soluble fusion protein comprises a soluble CD137L polypeptide fused to a polypeptide derived from the constant region of an antibody. Multimers resulting from formation of interchain disulfide bonds between the antibody-derived moieties of such fusion proteins are provided. If two different fusion proteins are made, one comprising an inventive protein fused to the heavy chain of an antibody and the other comprising an inventive protein fused to the light chain of an antibody, it is possible to form oligomers comprising as many as four soluble CD137L polypeptides. Methods of making such immunoglobulin fusion proteins are well-known in the art, and are described, for example, in U.S. Pat. No. 5,783,665, issued Jul. 21, 1998, the relevant disclosure of which is incorporated by reference herein.

In another embodiment, a soluble fusion protein comprises a zipper peptide that facilitates formation of oligomeric forms of CD137L/4-1BBL. Useful zipper peptides (and methods of making fusion proteins comprising the same) are disclosed in U.S. Pat. No. 5,716,825, issued Feb. 10, 1998, the relevant disclosure of which is incorporated by reference herein.

Protein fusions can comprise peptides added to facilitate purification or identification (referred to as ‘tags’) of CD137L polypeptides and homologs. One such tag peptide is poly-His; another tag peptide is the FLAG® peptide (Hopp et al., Bio/Technology 6:1204, 1988), which is highly antigenic and provides an epitope reversibly bound by a specific monoclonal antibody, enabling rapid assay and facile purification of expressed recombinant protein. A murine hybridoma designated 4E1 produces a monoclonal antibody that binds the FLAG® peptide in the presence of certain divalent metal cations, as described in U.S. Pat. No. 5,011,912, hereby incorporated by reference. The 4E11 hybridoma cell line has been deposited with the American Type Culture Collection under accession no. HB 9259. Monoclonal antibodies that bind the FLAG® peptide are available from Eastman Kodak Co., Scientific Imaging Systems Division, New Haven, Conn. The FLAG® polypeptide is also specifically cleaved by bovine mucosal enterokinase, allowing removal of the peptide from the purified protein; fusion proteins capped with such peptides may also be resistant to intracellular degradation in E. coli.

Additional, useful tag proteins include green fluorescent protein (GFP; Chalfie et al., Science 263:802, 1994); an N-terminal peptide that contains recognition sites for a monoclonal antibody, a specific endopeptidase, and a site-specific protein kinase (PKA; Blanar and Rutter, Science 256:1014, 1992); birA (Altman et al., Science 274:94, 1996); and glutathione S transferase (GST; Smith and Johnson, Gene 67:31, 1988).

Other useful proteins include CD137L polypeptides encoded by DNAs capable of hybridizing to the DNA of SEQ ID NOs:1 or 3 under moderately stringent conditions (prewashing solution of 5×SSC, 0.5% SDS, 1.0 mM EDTA (pH 8.0) and hybridization conditions of 50 degrees C., 5×SSC, overnight) to the DNA sequences encoding CD137L polypeptides, or more preferably under stringent conditions (for example, hybridization in 6×SSC at 63 degrees C. overnight; washing in 3×SSC at 55 degrees C.), and other sequences which are degenerate to those which encode CD137L. In one embodiment, CD137L polypeptides are at least about 70% identical in amino acid sequence to the amino acid sequence of native CD137L polypeptides as set forth in SEQ ID NOs:2 or 4. In a preferred embodiment, CD137L polypeptides are at least about 80% identical in amino acid sequence to the native form of CD137L; more preferred polypeptides are those that are at least about 90% identical to native CD137L; most preferred polypeptides are those that are at least about 95% identical to native CD137L.

Percent identity may be determined by visual inspection and mathematical calculation, or by using a computer program. Preferably, the comparison is done using a computer program. An exemplary, preferred computer program is the Genetics Computer Group (GCG; Madison, Wis.) Wis. package version 10.0 program, ‘GAP.’ The preferred default parameters for the ‘GAP’ program includes: (1) The GCG implementation of the previously stated comparison matrixes for nucleotides and amino acids; (2) a penalty of 30 for each gap and an additional penalty of 1 for each symbol in each gap for amino acid sequences, or penalty of 50 for each gap and an additional penalty of 3 for each symbol in each gap for nucleotide sequences; (3) no penalty for end gaps; and (4) no maximum penalty for long gaps. Other programs used by one skilled in the art of sequence comparison may also be used.

Antibodies

Antibodies that are immunoreactive with CD137/4-1BB are also useful in the inventive methods, particularly antibodies that are agonistic (that is, antibodies that bind CD137/4-1BB and transduce a signal thereby). Both polyclonal and monoclonal antibodies may be prepared by conventional techniques, and will be useful in the present invention. See, for example, Monoclonal Antibodies, Hybridomas: A New Dimension in Biological Analyses, Kennet et al. (eds.), Plenum Press, New York (1980); and Antibodies: A Laboratory Manual, Harlow and Land (eds.), Cold Spring Harbor Laboratory Press, Cold Spring Harbor, N.Y., (1988).

Hybridoma cell lines that produce monoclonal antibodies specific for CD137/4-1BB are also contemplated herein. Such hybridomas may be produced and identified by conventional techniques. One method for producing such a hybridoma cell line comprises immunizing an animal with a polypeptide or a DNA encoding a polypeptide; harvesting spleen cells from the immunized animal; fusing said spleen cells to a myeloma cell line, thereby generating hybridoma cells; and identifying a hybridoma cell line that produces a monoclonal antibody that binds the polypeptide. The monoclonal antibodies may be recovered by conventional techniques.

A useful hybridoma cell line was deposited with the American Type Culture Collection, in Manassas, Va. on Nov. 28, 2001 and given accession number PTA-3885. Antibody 4-1BBm6, produced by this hybridoma cell line, binds to murine CD137. Similar agonistic antibodies, including antibodies that bind human CD137, are obtained using methods that are known in the art. For example, antibodies that bind human CD137 may be isolated by utilizing a human CD137 immunogen in conventional methods for preparing monoclonal antibodies. Agonistic CD137 antibodies will have the ability to stimulate the proliferation of T cells, as determined by a proliferation assay such as those described in Example 1 herein, or that are well-known in the art.

Alternatively, recombinant methods such as those described herein can be utilized to isolate the DNA encoding antibody 4-1BBm6 or a similar, agonistic antibody (for example, an antibody that binds human CD137). The DNA can then be manipulated by methods that are known in the art to produce an antibody having desired characteristics, including the ability to bind human CD137 with high affinity. Useful techniques include chain shuffling, which has been used to prepare high affinity antibodies against the hapten 2-phenyloxazol-5-one, as described by Marks et al. (BioTechnology, 10:779, 1992), and molecular evolution of the CDRs, as described by Schier et al. (J. Mol. Biol. 263:551, 1996).

For use of antibodies as in vivo diagnostic or therapeutic agents in humans, it is often desirable to use a human antibody, or an antibody that is less likely to generate an immune response than a non-human antibody. Many techniques have been developed to facilitate production of such antibodies. For example, techniques developed for the production of “chimeric” (i.e., having portions derived from different species) antibodies (Takeda et al., Nature, 314:452, 1985; Morrison et al., Proc. Natl. Acad. Sci. USA 81:6851, 1984; Boulianne et al., Nature, 312:643, 1984; Neuberger et al., Nature, 314:268, 1985) by splicing the genes from a mouse antibody molecule of appropriate antigen specificity together with genes from a human antibody molecule of appropriate biological activity can be used.

A chimeric monoclonal antibody may comprise the variable region of a non-human antibody (or just the antigen binding site thereof) and a constant region derived from a human antibody. Alternatively, a chimeric antibody can comprise the antigen binding site of a non-human monoclonal antibody and a variable region fragment (lacking the antigen-binding site) derived from a human antibody. However, such chimeric antibodies may still contain enough residues from the non-human species from which the antibody was derived to render it immunogenic in humans.

Procedures for the production of engineered monoclonal antibodies that are less likely to generate an immune response in a human include those described in Riechmann et al. (Nature 332:323, 1988), Liu et al. (PNAS 84:3439, 1987), Larrick et al. (Bio/Technology 7:934, 1989), and Winter and Harris (TIPS 14:139, Can, 1993). Such antibodies are referred to as “humanized;” generally, some residues in the hyper-variable or complementarity determining regions (CDRs), and sometimes selected framework (FR) residues, in a human antibody are substituted by residues from analogous sites in other (i.e., rodent) antibodies. Useful techniques for humanizing antibodies are also discussed in U.S. Pat. No. 6,054,297, issued Apr. 25, 2000.

In addition, human antibodies can be produced in animals that have been genetically manipulated to have human immunoglobulin genes (transgenic animals). Procedures to generate such human antibodies transgenically can be found in GB 2,272,440, U.S. Pat. Nos. 5,569,825 and 5,545,806 and related patents claiming priority therefrom, all of which are incorporated by reference herein. Techniques for creating such human or humanized antibodies are also well known and are commercially available from, for example, Medarex Inc. (Princeton, N.J.) and Abgennix Inc. (Fremont, Calif.). Preferably, for use in humans, the antibodies are human or humanized.

Techniques described for the production of single chain antibodies (U.S. Pat. No. 4,946,778; Bird, 1988, Science 242:423; Huston et al., 1988, Proc. Natl. Acad. Sci. USA 85:5879; and Ward et al., 1989, Nature 334:544) can also be adapted to produce single chain antibodies against CD137 and another antigen (or two different epitopes of CD137). Single chain antibodies are formed by linking the heavy and light chain fragments of the Fv region via an amino acid bridge, resulting in a single chain polypeptide. The single chain antibodies against against CD137 and another antigen (or two different epitopes of CD137) may be concatamerized in either order. Once desired single chain antibodies are identified (for example, from a phage-display library), those of skill in the art can further manipulate the DNA encoding the single chain antibody(ies), for example, to add all or a portion of an Fc region.

The foregoing antibodies can be further manipulated using methods that are known in the art to generate recombinant antibodies (for example, using the methods of Marks et al., supra and/or Schier et al., supra, as well as to prepare single chain Fv or scFv, Fab, F(ab′)2, diabodies, and/or triabodies, as well as antibodies having a desired Fc, for example IgA, IgG1, IgG2, IgG3, IgG4, IgM, IgE, IgD, and IgG4 having a mutation in a hinge region that alleviates a tendency to form intra-H chain disulfide bonds). Antigen-binding fragments of the antibodies, which may be produced by conventional techniques, are also useful in the present invention. Examples of such fragments include, but are not limited to, Fab and F(ab′)2 fragments. Antibody fragments and derivatives produced by genetic engineering techniques are also provided, including single chain Fv or scFv, as well as diabodies and triabodies.

Additional, useful antibodies include bispecific antibodies. Bispecific antibodies bind a first epitope with a first antigen-binding site, and a second epitope with a second antigen binding site. The two epitopes may be present on the same polypeptide (i.e., two different epitopes present on CD137/4-1BB). Alternatively, the epitopes may be from two different polypeptides. Such bispecific antibodies have the property of bringing into close proximity the two polypeptides to which they bind. Thus, bispecific antibodies that are useful in the present inventive methods comprise antibodies that bind CD137/4-1BB via a first antigen binding domain, and bind another member of the TNFR/NGFR family (for example, TNF-RI/p55, TNF-RII/p75, TNF-RIII/TNF-RP, poxvirus proteins T2 and A53R, Ox-40, CD40, CD30, CD27, p75(NGFR), CAR1, Fas, RANK, AIR, DR3/APO3/WSL-1/TRAMP/LARD, DR4, DR5/APO2/TRAIL-R2/TRICK2/KILLER, DcR1/LIT/TRID/TRAIL-R3, DcR2/TRUNDD/TRAIL-R4) via the second antigen binding domain. In one embodiment, a bispecific antibody binds CD137 via a first antigen binding domain, and Ox-40 via a second antigen binding domain. In another embodiment, a bispecific antibody binds CD137/4-1BB via a first antigen binding domain, and an epitope bound by Xolair™ via a second antigen binding domain. It is also understood that the techniques discussed herein can be used to prepare antibodies that have specificity for more than two epitopes.

A variety of bispecific antibodies have been prepared, and found useful both in vitro and in vivo (see, for example, U.S. Pat. No. 5,807,706, issued Sep. 15, 1998). Numerous methods of preparing bispecific antibodies are known in the art. One such method involves the use of hybrid-hybridomas as described by Milstein and Cuello (Nature 305:537; 1983). When two hybridoma cells are fused, the resulting cell is referred to as a “quadroma;” a “trioma” is formed by the fusion of a hybridoma and a lymphocyte (see, for example, U.S. Pat. No. 4,474,893, issued Oct. 2, 1984). At least a portion of the antibodies produced by hybrid hybridoma cells will be bispecific. U.S. Pat. No. 6,106,833, issued Aug. 22, 2000, also discloses methods of making hybrid hybridoma and/or trioma cells that are useful in the production of bispecific antibodies.

U.S. Pat. No. 6,060,285, issued May 9, 2000, discloses a process for the production of bispecific antibodies in which at least the genes for the light chain and the variable portion of the heavy chain of an antibody having a first specificity are transfected into a hybridoma cell secreting an antibody having a second specificity. When the transfected hybridoma cells are cultured, the bispecific antibodies are produced and may be isolated by various means known in the art.

Other investigators have used chemical coupling of antibody fragments to prepare antigen-binding molecules having specificity of two different antigens (Brennan et al., Science 229:81 1985; Glennie et al., J. Immunol. 139:2367, 1987). U.S. Pat. No. 6,010,902, issued Jan. 4, 2000, also discusses techniques known in the art by which bispecific antibodies can be prepared, for example by the use of heterobifunctional cross-linking reagents such as GMBS (maleimidobutryloxy succinimide) or SPDP (N-succinimidyl 3-(2-pyridyldithio)propionate) [see, e.g., Hardy, “Purification And Coupling Of Fluorescent Proteins For Use In Flow Cytometry”, Handbook Of Experimental Immunology, Volume 1, Immunochemistry, Weir et al. (eds.), pp. 31.4-31.12 4th Ed., (1986)].

The ability to produce antibodies via recombinant means has facilitated production of bispecific antibodies. Kostelny et al. utilized the leucine zipper moieties from the Fos and Jun proteins (which preferentially form heterodimers) to produce bispecific antibodies able to bind both the cell surface molecule CD3 and the receptor for Interleukin-2 (J. Immunol. 148:1547; 1992). Moreover, single-chain variable fragments (sFvs) have been prepared by covalently joining two variable domains; the resulting antibody fragments can form dimers or trimers, depending on the length of a flexible linker between the two variable domains (Kortt et al., Protein Engineering 10:423; 1997).

U.S. Pat. No. 5,582,996, issued Dec. 10, 1996, discloses the use of complementary interactive domains (such as leucine zipper moieties or other lock and key interactive domain structures) to facilitate heterodimer formation in the production of bispecific antibodies. The complementary interactive domain(s) may be inserted between an Fab fragment and another portion of a heavy chain (i.e., CH1 or CH2 regions of the heavy chain). The use of two different Fab fragments and complementary interactive domains that preferentially heterodimerize will result in bispecific antibody molecules. Cysteine residues may be introduced into the complementary interactive domains to allow disulphide bonding between the complementary interactive domains and stabilize the resulting bispecific antibodies.

Tetravalent, bispecific molecules can be prepared by fusion of DNA encoding the heavy chain of an F(ab′)2 fragment of an antibody with either DNA encoding the heavy chain of a second F(ab′)2 molecule (in which the CHI domain is replaced by a CH3 domain), or with DNA encoding a single chain FV fragment of an antibody, as described in U.S. Pat. No. 5,959,083, issued Sep. 28, 1999. Expression of the resultant fusion genes in mammalian cells, together with the genes for the corresponding light chains, yields tetravalent bispecific molecules having specificity for selected antigens.

Bispecific antibodies can also be produced as described in U.S. Pat. No. 5,807,706, issued Sep. 15, 1998. Generally, the method involves introducing a protuberance (constructed by replacing small amino acid side chains with larger side chains) at the interface of a first polypeptide and a corresponding cavity (prepared by replacing large amino acid side chains with smaller ones) in the interface of a second polypeptide. The protuberance and cavity can be made by synthetic means such as altering the nucleic acid encoding the polypeptides or by peptide synthesis, and are positioned so as to promote heteromultimer formation and hinder homomultimer formation.

Therapeutic Methods and Administration of Agonists

Methods provided herein comprise administering a CD137 agonist to a patient, thereby inducing a CD137-mediated biological response that plays a role in a particular condition. Treatment encompasses alleviation of at least one symptom of a disorder, or reduction of disease severity, and the like. An agonist need not effect a complete “cure”, or eradicate every symptom or manifestation of a disease, to constitute a viable therapeutic agent. As is recognized in the pertinent field, drugs employed as therapeutic agents may reduce the severity of a given disease state, but need not abolish every manifestation of the disease to be regarded as useful therapeutic agents. One embodiment of the invention is directed to a method comprising administering to a patient a CD137 agonist in an amount and for a time sufficient to induce a sustained improvement over baseline of an indicator that reflects the severity of the particular disorder.

As is understood in the pertinent field, CD137 agonists are administered to a patient in a manner appropriate to the indication. Agonists may be administered by any suitable technique, including but not limited to parenterally, topically, or by inhalation. If injected, the agonist can be administered, for example, via intra-articular, intravenous, intramuscular, intralesional, intraperitoneal or subcutaneous routes, by bolus injection, or continuous infusion. Localized administration, e.g. at a site of disease or injury is contemplated, as are transdermal delivery and sustained release from implants. Delivery by inhalation includes, for example, nasal or oral inhalation, use of a nebulizer, inhalation of the agonist in aerosol form, and the like. Other alternatives include eyedrops; oral preparations including pills, syrups, lozenges or chewing gum; and topical preparations such as lotions, gels, sprays, and ointments.

Advantageously, agonists are administered in the form of a composition comprising at least a CD137 agonist and one or more additional components such as a physiologically acceptable carrier, excipient or diluent. The present invention provides such compositions comprising an effective amount of a CD137 agonist, for use in the methods provided herein.

The compositions contain agonist(s) in any of the forms described herein. The agonist may be a whole antibody or an antigen-binding fragment or engineered derivative thereof, for example. For compositions containing CD137L, the ligand may be any of the fragments, variants, or oligomers of the protein described herein or in U.S. Pat. No. 5,674,704, for example.

Compositions may, for example, comprise an agonist together with a buffer, antioxidant such as ascorbic acid, low molecular weight polypeptide (such as those having fewer than 10 amino acids), protein, amino acid, carbohydrate such as glucose, sucrose or dextrins, chelating agents such as EDTA, glutathione, and other stabilizers and excipients. Neutral buffered saline or saline mixed with conspecific serum albumin are examples of appropriate diluents. In accordance with appropriate industry standards, preservatives such as benzyl alcohol may also be added. The composition may be formulated as a lyophilizate using appropriate excipient solutions (e.g., sucrose) as diluents. Suitable components are nontoxic to recipients at the dosages and concentrations employed. Further examples of components that may be employed in pharmaceutical formulations are presented in Remington's Pharmaceutical Sciences, 16^{th} Ed., Mack Publishing Company, Easton, Pa., 1980.

Kits for use by medical practitioners include a CD137 agonist and a label or other instructions for use in treating any of the conditions discussed herein. The kit preferably includes a sterile preparation of one or more CD137 agonists, which may be in the form of a composition as disclosed above, and may be in one or more vials.

Dosages and the frequency of administration may vary according to such factors as the route of administration, the particular agonist employed, the nature and severity of the disease to be treated, whether the condition is acute or chronic, and the size and general condition of the patient. Appropriate dosages can be determined by procedures known in the pertinent art, e.g. in clinical trials that may involve dose escalation studies.

An agonist may be administered once, or repeatedly. In particular embodiments, the agonist is administered over a period of at least a month or more, e.g., for one, two, or three months or even indefinitely. For treating chronic conditions, long-term treatment is generally most effective. However, for treating acute conditions, administration for shorter periods, e.g. from one to six weeks, may be sufficient. In general, the agonist is administered until the patient manifests a medically relevant degree of improvement over baseline for the chosen indicator or indicators.

Particular embodiments of the present invention involve administering an agonist at a dosage of from about 1 ng/kg/day to about 10 mg/kg/day, more preferably from about 500 ng/kg/day to about 5 mg/kg/day, and most preferably from about 5 micrograms/kg/day to about 2 mg/kg/day, to a patient. In additional embodiments, an agonist is administered one time per week, two times per week, or three or more times per week, to treat the medical disorders disclosed herein. If injected, the effective amount of agonist per dose may range from 1-20 mg/m², and preferably is about 5-12 mg/m². Alternatively, a flat dose may be administered; the amount may range from 5-100 mg/dose. One range for a flat dose is about 20-30 mg per dose. In one embodiment of the invention, a flat dose of 25 mg/dose is repeatedly administered by injection. If a route of administration other than injection is used, the dose is appropriately adjusted in accordance with standard medical practices. One example of a therapeutic regimen involves injecting a dose of about 20-30 mg of CD137 agonist one to three times per week over a period of at least three weeks, though treatment for longer periods may be necessary to induce the desired degree of improvement. For pediatric patients (age 4-17), one suitable regimen involves the subcutaneous injection of 0.4 mg/kg, up to a maximum dose of 25 mg of CD137 agonist, administered two or three times per week.

Particular embodiments of the methods provided herein involve subcutaneous injection of from 0.5 mg to 10 mg, preferably from 3 to 5 mg, of a CD137 agonist, once or twice per week. Another embodiment is directed to pulmonary administration (e.g., by nebulizer) of 3 or more mg of a soluble CD137 agonist once a week. Examples of therapeutic regimens provided herein comprise subcutaneous injection of CD137 agonist once a week, at a dose of 1.5 to 3 mg, to treat asthma, atopic dermatitis or allergy. Weekly administration of CD137 agonist is continued until symptoms subside. Treatment may resume as needed, or, alternatively, maintenance doses may be administered.

An agonist is administered to the patient in an amount and for a time sufficient to induce an improvement, preferably a sustained improvement, in at least one indicator that reflects the severity of the disorder that is being treated. Various indicators that reflect the extent of the patient's illness may be assessed for determining whether the amount and time of the treatment is sufficient. Such indicators include, for example, clinically recognized indicators of disease severity, symptoms, or manifestations of the disorder in question. In most instances, an improvement is considered to be sustained if the patient exhibits the improvement on at least two occasions separated by two to four weeks. The degree of improvement generally is determined by the patient's physician, who may make this determination based on signs or symptoms, and who may also employ questionnaires that are administered to the patient, such as quality-of-life questionnaires developed for a given disease.

Particular embodiments of methods and compositions of the invention involve the use of two or more different CD137 agonists. In further embodiments, CD137 agonist(s) are administered alone or in combination with other agents useful for treating the condition with which the patient is afflicted. Examples of such agents include both proteinaceous and non-proteinaceous drugs. When multiple therapeutics are co-administered, dosages may be adjusted accordingly, as is recognized in the pertinent art. “Co-administration” and combination therapy are not limited to simultaneous administration, but include treatment regimens in which a CD137 agonist is administered at least once during a course of treatment that involves administering at least one other therapeutic agent to the patient (i.e., a CD137 agonist and another agent or agents may be administered concurrently, consecutively, or in the form of a single composition comprising two or more agents as active ingredients).

Examples of other agents that may be co-administered with CD137 agonists are other antibodies, cytokines, or cytokine receptors, which are chosen according to the particular condition to be treated. Alternatively, non-proteinaceous drugs that are useful in treating one of the particular conditions discussed above may be co-administered with a CD137 agonist.

For treating IgE-mediated conditions, a CD137 agonist may be co-administered with an IgE antagonist. One example is an anti-IgE antibody. Humanized anti-IgE monoclonal antibodies are described in Presta et al. (J. Immunol. 151(5):2623-2632, 1993) and Adelroth et al. (J. Allergy Clin. Immunol. 106(2):253-259, 2000), for example. This antibody, known as Omalizumab or Xolair™, (Novartis International AG, Basel, Switzerland) has been used for the treatment of severe allergic asthma (Busse W. W., J Allergy Clin Immunol 108:184-, 2001), seasonal allergic rhinitis (Casale T. B., Am J Respir Crit Care Med. 164:S18; 2001), and other allergic diseases (Soler, M.; Int J Clin Pract. 55:480, 2001).

CD137 agonists may be co-administered with cytokine antagonists (for example, an IL-4 and/or an IL-5 antagonist), which may be a molecule that interferes with the binding of the cytokine to its respective receptor, such as an anti-cytokine or anti-receptor antibody (e.g., a human or humanized monoclonal antibody), a soluble form of the receptor, a small molecule that interferes with the binding of the cyoktine and receptor, or a molecule that interferes with signal transduction via the receptor. In particular, IL-4 and IL-5 have been implicated in mediating allergic responses, and IL-9 and IL-13 have been implicated in asthma. Thus, administration of antagonist(s) of one or more of these cytokines along with a CD137 agonist is contemplated for treatment of allergic diseases, including but not limited to allergic asthma.

IL-5 antagonists include a dimeric peptide that binds to the IL-5 receptor alpha/beta heterodimer complex (England et al., Proc Natl Acad Sci USA 97:6862, 2001), and humanized antibodies to IL-5 (Leckie et al., Lancet 356:2144, 2000). IL-9 antagonists include receptors such as those described in WO 93/18047 and U.S. Pat. Nos. 5,789,237 and 5,962,269, which are hereby incorporated by reference herein. Inhibitors of IL-4 include soluble IL-4 receptor (described in U.S. Pat. No. 5,599,905, issued Feb. 4, 1997; hereby incorporated by reference), as well as antibodies that bind IL-4 receptor (IL-4R) and inhibit binding of IL-4 thereto (described in U.S. Pat. No. 5,599,905, and in PCT application WO 01/923402; hereby incorporated by reference); such antibodies may also inhibit binding of IL-13 to IL-4R complexes.

CD137 agonists may be employed in conjunction with other agent(s) in treating the particular conditions discussed above (i.e., a CD137 agonist and another agent or agents may be administered concurrently, consecutively, or in the form of a single composition comprising two or more agents as active ingredients). For example, drugs currently employed in treating the conditions may be co-administered with one or more CD137 agonists. Several types of cytokine-directed therapies for asthma patients are discussed by P. J. Barnes (J Allergy Clin Immunol 108(2 Suppl):S72-6, 2001), and include inhibitors of pro-inflammatory cytokines. For treating asthma, a CD137 agonist may be co-administered with other anti-asthma medications, such as inhaled corticosteroids, beta agonists, leukotriene antagonists, xanthines, fluticasone, salmeterol, albuterol, non-steroidal agents such as cromolyn, and the like. CD137 agonists may be co-administered with other anti-allergy medications to treat allergic reactions.

One embodiment of the present invention is directed to co-administration of a CD137 agonist and fluticasone and salmeterol to treat a disorder such as asthma. Compositions comprising a CD137 agonist, fluticasone, and salmeterol are provided herein. Advair Diskus® (Glaxo Wellcome) comprises fluticasone propionate and salmeterol xinafoate. For treating asthma, Advair Diskus® and the CD137 agonist preferably are delivered by inhalation.

One method provided herein for treating asthma comprises administering a CD137 agonist and interferon-γ to a human who has asthma. Another method for treating asthma comprises co-administering a CD137 agonist, IFN-γ, and one or more cytokine antagonists to a human who has asthma. In one embodiment, the CD137 agonist and IFN-γ are co-administered to an asthmatic, together with an antibody that functions as an antagonist of both IL-4 and IL-13. Such antibodies are described in PCT application WO 01/923402, supra.

Antagonists may be co-administered with one or more leukotriene receptor antagonists to treat disorders such as allergic inflammatory diseases, e.g., asthma, atopic dermatitis and allergies. Examples of leukotriene receptor antagonists include but are not limited to montelukast, pranlukast, and zafirlukast. Drugs that function as 5-lipoxygenase inhibitors may be co-administered with an IL-4 antagonist to treat asthma.

Methods provided herein comprise administering a CD137 agonist to Churg-Strauss Syndrome patients, optionally along with one or more of the following: IL-4 antagonist(s), IL-5 antagonist(s), IL-13 antagonist(s) or IgE antagonist(s). One example of such a method involves co-administering a CD137 agonist and two or more agents, for example, IL-4 antagonist(s) and IL-5 antagonist(s), to a Churg-Strauss Syndrome patient. In another embodiment, the additional agents IL-4 antagonist(s) and IgE antagonist(s) are co-administered to the patient with a CD137 agonist. In yet another embodiment, the additional agents co-administered to the patient include IL-4 antagonist(s) and IL-13 antagonist(s).

Additional useful agents for co-administration with a CD137 agonist include agents that modulate an inflammatory and/or autoimmune response. One such agent is an IL-17 antagonist. Any suitable IL-17 antagonist may be employed, including but not limited to an IL-17 receptor (preferably soluble forms thereof), IL-17 receptor antagonists, antibodies directed against IL-17 or an IL-17 receptor, other proteins that interfere with the binding of IL-17 to an IL-17 receptor, and compounds that inhibit IL-17-mediated signal transduction. An IL-17 receptor, including soluble forms thereof and oligomers thereof, is described in WO 96/29408, hereby incorporated by reference. An alternative method provided herein comprises administering an IL-17 antagonist to treat a patient with benign prostate hyperplasia.

Another suitable agent include that modulates an inflammatory and/or autoimmune response is a TNF antagonist. TNF antagonists that may be employed for coadministration with a CD137 agonist, include, but are not limited to, a TNF receptor (preferably soluble forms thereof), fusion proteins comprising a TNF receptor (or comprising the TNF-binding portion of a TNF receptor), TNF receptor antagonists, antibodies directed against TNF or a TNF receptor, other proteins that interfere with the binding of TNF to a TNF receptor, and compounds that inhibit TNF-mediated signal transduction. Further examples of TNF inhibitors are the drugs thalidomide and pentoxyfylline. The TNF receptor protein known as p75 or p80 TNF-R preferably is employed. A preferred TNF antagonist is a soluble human TNF receptor (sTNF-R) in dimeric form, such as dimers of sTNF-R/Fc fusion proteins. One such dimer is etanercept (Enbrel®, Immunex Corporation, Seattle, Wash.). p75/p80 TNF-R, including soluble fragments and other forms thereof, is described in WO 91/03553, hereby incorporated by reference herein.

In accordance with the present invention, a CD137 agonist is co-administered with a TNF antagonist to treat any condition in which undesirable TNF-induced immune responses play a role, such as inflammation. One method provided herein comprises co-administering a CD137 agonist and a TNF antagonist to a patient with inflammatory bowel disease, Crohn's disease, or ulcerative colitis. Other embodiments are directed to a method comprising co-administering a CD137 agonist and a TNF antagonist to a patient who has Kawasaki Disease, autoimmune hemolytic anemia, autoimmune uveoretinitis, autoimmune lymphoproliferative syndrome, Sjogren's syndrome, chronic fatigue syndrome, or hepatotoxicity induced by a drug such as diclofenac.

Suitable dosages of etanercept (Enbrel®, Immunex Corporation, Seattle, Wash.) will vary according to the nature of the disease to be treated, disease severity, the size of the patient (e.g., adult or child), and other factors, as is recognized in the pertinent field. In one embodiment of the methods provided herein, Enbrel® is administered twice a week by subcutaneous injection at a dose of from 1 to 25 mg. One embodiment of a pediatric dosage is 0.4 mg/kg. Particular methods provided herein comprise co-administration of an IL-4 antagonist and Enbrel® to a patient has autoimmune lymphoproliferative syndrome or Sjogren's syndrome, wherein Enbrel® is given by subcutaneous injection at a dose of from 1 to 25 mg.

Another agent that modulates an inflammatory and/or autoimmune response is an IL-1 antagonist. A preferred IL-1 antagonist is a soluble form of type II IL-1 receptor, which is described in U.S. Pat. No. 5,350,683. Other IL-1 antagonists are known in the art, and include Kineret™ (Amgen Inc., Thousand Oaks, Calif.), a recombinant form of the IL-1 receptor antagonist.

A number of cytokine agonists and/or antagonists and other agents/drugs are disclosed herein as being useful for combination therapy (e.g., co-administration with a CD137 agonist) in treating particular diseases. It is to be understood that such agents, or drugs, also find use as single agents in treating those diseases. It also is to be understood that disclosure of methods involving administration of an agonist or antagonist to a particular cytokine, to treat a disease, encompasses administration of one type of agonist or antagonist, and also encompasses administration of two or more different agonists and/or antagonists for that cytokine, unless specified otherwise.

The embodiments within the specification and the following examples provide an illustration of embodiments of the invention and should not be construed to limit the scope of the invention. Those of ordinary skill in the art will recognize that many other embodiments are encompassed by the claimed invention.

### EXAMPLE 1

### Effect of CD137 Agonists (4-1BB Agonists) on T-Cell Proliferation

(a) Peripheral Blood T-Cells

The ability of a CD137 agonist to costimulate T-cell proliferation is assessed in a 3 day tritiated thymidine-incorporation assay. The assay procedure was generally as described by Goodwin et al. (Cell 73:447, 1993). Briefly, human peripheral blood T-cells are isolated and cultured with a titration of CD137 agonist, in the presence of suboptimal PHA (0.1%) as a costimulus. After 3 days, cultures are pulsed with [³H] thymidine and incorporated radioactivity is assessed approximately six hours later.

CD137 agonists will markedly enhance T-cell proliferation induced by sub-optimal PHA, whereas agents that do not act as CD137 agonists will have no effect.

(b) T-Cell Clone

The effect of a CD137 agonist on a long term cultured T-cell clone may also be analyzed. Chronically activated T-cells, such as long-term grown T-cell clones (TCC), are induced to undergo programmed cell death when stimulated with mitogens such as anti-CD3 mAb or PHA in the absence of antigen-presenting cells (Wesselborg et al., J. Immunol. 150:4338, 1993). TCC that express CD137 are used to assess the effect of CD137 agonists on the growth of the alloreactive CD4⁺ human T-cell clone designated PL-1.

PL-1 cells are cultured for 3 days in the presence or absence of suboptimal PHA (0.1%) or another mitogen as costimulus and CD137 agonist (or control). Viability is determined by trypan blue exclusion. In the presence of PHA, addition of CD137 agonist will reduce the viability of PL-1 cells by enhancing the activation-induced cytolysis of the PL-1 cells.

### EXAMPLE 2

### Effect of CD137 Agonists (4-1BB Agonists) on Eosinophils

The activity of a CD137 agonist on eosinophils is assessed by evaluating expression of CD137 and determination of eosinophil death and/or apoptosis. Eosinophils are obtained substantially as described by Hansel et al. (J Immunol Methods 145:105, 1991). Briefly, a peripheral blood granulocyte preparation, containing neutrophils and eosinophils, is incubated with superparamagnetic particles coupled to a monoclonal antibody against CD16, a molecule present on neutrophils but not on eosinophils. Magnetically labelled neutrophils are retained on columns with a ferromagnetic matrix, and a highly-enriched, purified population of eosinophils is obtained. Expression of CD137 is assessed either by evaluating cells for the expression of CD137 protein, for example by FACS analysis using an antibody to CD137, or by expression of CD137 mRNA, using a probe or probes derived from the coding sequence for CD137 (SEQ ID NO:1 or 3). Eosinophils are cultured in the presence or absence of cytokines substantially as described by Heinisch et al., J. Allergy Clin. Immunol. 108:21 (2001). The effect of CD137 agonists on eosinophils is determined by assesing the eosinophils for death and/or apoptosis by any of several methods that are known in the art, including those described by Heinisch et al., supra, Simon et al., J. Immunol. 158:3902 (1997), Wedi et al., J. Allergy Clin. Immunol. 100:536 (1997), Vignola et al., J. Allergy Clin. Immunol. 103:563 (1999) and Kankaanranta et al., J. Allergy Clin. Immunol. 106:77 (2000).

What is claimed is:

1. A method for treating a condition mediated by IgE, comprising administering a CD137 agonist to a mammal afflicted with such a condition.

2. The method of claim 1, wherein the CD137 agonist is selected from the group consisting of CD137 ligand (CD137L) and agonistic antibodies to CD137.

3. The method of claim 2, wherein the CD137 agonist is a CD137L polypeptide selected from the group consisting of:
(a) a polypeptide comprising amino acids x through y of SEQ ID NO:2, wherein x is selected from the group consisting of amino acids 104, 105, 106, 107, 108 and 109 of SEQ ID NO:2, and y is selected from the group consisting of amino acids 304, 305, 306, 307, 308 and 309 of SEQ ID NO:2;
(b) a polypeptide comprising amino acids x through y SEQ ID NO:4, where x is selected from the group consisting of amino acids 49, 50, 51, 52, 53, and SEQ ID NO:4 and y is selected from the group consisting of amino acids 249, 250, 251, 252, 253 and 254 of SEQ ID NO:4;
(c) CD137L polypeptides that are at least about 80% identical in amino acid sequence to the polypeptides of (a) or (b); and
(d) fragments of the aforementioned CD137L polypeptides that are CD137 agonists.

4. The method of claim 2, wherein the CD137 agonist is an agonistic antibody to CD137.

5. The method of claim 4 wherein the antibody to CD137 is selected from the group consisting of:
(a) an antibody produced by hybridoma cell line 4-1BBm6, deposited with the American Type Culture Collection, in Manassas, Va. on Nov. 28, 2001 and given accession number PTA-3885;
(b) an antibody derived from the hybridoma cell line of (a); and
(c) derivative and mutants of the aforementioned antibodies, including scFv, Fab, F(ab′)2, diabodies, triabodies, IgA, IgG1, IgG2, IgG3, IgG4, IgM, IgE, IgD, and IgG4 having a mutation in a hinge region that alleviates a tendency to form intra-H chain disulfide bonds.

6. The method of claim 2 wherein the condition mediated by IgE is selected from the group consisting of asthma, atopic dermatitis, allergy, and combinations thereof.

7. The method of claim 6, wherein the condition mediated by IgE is characterized by delayed eosinophil apoptosis.

8. The method of claim 6 wherein the condition is selected from the group consisting of nasal polyps and hypereosinophilic syndrome.

9. The method of claim 2 wherein the CD137 agonist is co-administered with an agent that antagonizes a cytokine selected from the group consisting of IL-4, IL-5, IL-9, IL-13, and combinations thereof.

10. The method of claim 9 wherein the CD137 agonist is an antibody to CD137

11. The method of claim 2 wherein the CD137 agonist is an antibody to CD137 and the conditioned mediated by IgE is asthma.

12. The method of claim 2 wherein the CD137 agonist is co-administered with an anti-IgE antibody.

13. The method of claim 12 wherein the CD137 agonist is an antibody to CD137.

14. The method of claim 3 wherein the CD137 ligand is co-administered with an anti-IgE antibody.

15. The method of claim 14 wherein the IgE mediated condition is asthma.
